(19) United States
(12) Patent Application Publication
(10) Pub. No.: US 2026/0208163 A1
(43) Pub. Date: Jul. 23, 2026

(54) CATALYST FOR PARAFFIN DEHYDROGENATION

(71) Applicant: SABIC GLOBAL TECHNOLOGIES B.V., Bergen op Zoom (NL)

(72) Inventors: Biju Maippan DEVASSY, Bangalore (IN); Vinod Sankaran NAIR, Bangalore (IN); Velayutham SARAVANAN, Hosur (IN); Ritesh NANDY, Bangalore (IN)

(21) Appl. No.: 19/141,619

(22) PCT Filed: Dec. 18, 2023
(86) PCT No.: PCT/EP2023/086283

§ 371 Date: Jun. 20, 2025

Related U.S. Application Data
(60) Provisional application No. 63/476,641, filed on Dec. 22, 2022.

(51) Int. Cl. B01J 23/26; B01J 23/00; B01J 37/00; B01J 37/04; B01J 37/08; C07C 5/32

(52) U.S. Cl. CPC B01J 23/26; B01J 23/002; B01J 37/0009; B01J 37/04; B01J 37/08; C07C 5/322; B01J 2523/11; B01J 2523/12; B01J 2523/31; B01J 2523/67; C07C 2521/04; C07C 2523/26

(57) ABSTRACT

Methods of preparing and using alkane dehydrogenation catalysts to dehydrogenate alkanes are provided. The method for dehydrogenation of an alkane may include loading a reactor with a dehydrogenation catalyst produced by mixing a plurality of aluminum hydroxides, a water insoluble chromium (III) oxide source, and an alkali metal oxide source with an aqueous non-metal acidic solution to form a moldable mixture. The moldable mixture may be extruded to form extrudates. The extrudates may be dried and calcined to produce the alkane dehydrogenation catalyst containing from about 60 wt. % to about 90 wt. % of aluminum oxide, from about 10 wt. % to about 40 wt. % of trivalent chromium oxide, and from about 0.1 wt. % to about 5 wt. % of the alkali metal oxide. The method may further include supplying a feed containing alkanes through the reactor at a pressure of less than 1 atmosphere to dehydrogenate the alkanes.

### CROSS-REFERENCE TO RELATED APPLICATIONS

This application claims the benefit of priority to U.S. Provisional Patent Application No. 63/476,641, filed Dec. 22, 2022, the entire contents of which are hereby incorporated by reference in their entirety.

### TECHNICAL FIELD

The present disclosure generally relates to methods for dehydrogenation of an alkane as well as methods for preparing and using an alkane dehydrogenation catalyst. More specifically, the present disclosure relates to, among other embodiments, methods for the use and preparation of chromia-alumina dehydrogenation catalysts without the use of water-soluble chromium containing materials for the dehydrogenation of paraffins.

### BACKGROUND

Alkane dehydrogenation is a recognized process for the production of a variety of useful hydrocarbon products, such as isobutylene for conversion to MTBE, as well as isooctane and alkylates to supplement and enrich gasolines. There are several current catalytic processes useful for the catalytic dehydrogenation of light alkanes, including the Süd-Chemie CATOFIN® process, UOP's Oleflex® process, Phillips' Star™ process, and the Snamprogetti-Yarsintee process. The catalysts that are used in these processes are manufactured from two different groups of materials. The Süd-Chemie CATOFIN® process and the Snamprogetti-Yarsintee process utilize chromia-alumina catalysts. In contrast, the catalysts for the UOP and Phillips processes include supported precious metal platinum as catalysts.

Chromia-alumina dehydrogenation catalyst technology has been in use for many decades. The stability of dehydrogenation catalysts plays an important role in the overall efficiency of the dehydrogenation process. Because of the extreme temperature ranges at which the catalytic dehydrogenation procedure is conducted, the life expectancy of the catalyst is often limited. Thus, improving the stability of the catalyst translates into longer catalyst life, allowing for better catalyst utilization and ultimately resulting in lower consumption of the catalyst during the dehydrogenation process.

Chromia-alumina dehydrogenation catalysts are typically produced by the impregnation of an aluminum carrier with high-concentration chromic acid solution, which contains primarily hexavalent chromium (chromium (VI)), which is toxic and carcinogenic, and therefore highly undesirable for use on an industrial scale. Additionally, the catalyst preparation process involving the impregnation of an aluminum carrier with a chromium (III) salt requires multiple impregnation steps to arrive at a desirable chromium content. Each impregnation step requires intermediate drying and calcination, and so such a multi-impregnation method is time- and labor-intensive, and cost prohibitive relative to conventional preparation procedures involving chromium (VI)-containing materials. Accordingly, the Applicant has recognized that there exists a need for a simple, cost effective method for making a chromia-alumina dehydrogenation catalyst without the use of chromium (VI)-containing materials, and which exhibits good activity, improved stability, and for the dehydrogenation of lower paraffins.

### SUMMARY

To address shortcomings in the art, the Applicant has developed methods for the dehydrogenation of alkanes as well as methods for the preparation and use of alkane dehydrogenation catalysts. In addition to other embodiments, the Applicant has discovered cost-effective methods for making chromia-alumina dehydrogenation catalysts without the use of water soluble chromium containing sources. The presently disclosed dehydrogenation catalysts have good activity as well as improved stability. The presently disclosed chromia-alumina dehydrogenation catalysts are useful, among other uses, for the dehydrogenation of lower paraffin. Additionally, it has been unexpectedly discovered that supplying a feed containing alkanes through the reactor at a pressure of less than 1 atmosphere to dehydrogenate alkanes results in higher alkane dehydrogenation rates when using the catalysts prepared according to the presently disclosed methods.

According to one aspect of the present disclosure, a method for dehydrogenating an alkane is provided. In certain embodiments, the method may include loading a reactor with a dehydrogenation catalyst. The dehydrogenation catalyst may be prepared by mixing a plurality of aluminum hydroxides, a water insoluble chromium (III) oxide source, and an alkali metal oxide source with an aqueous non-metal acidic solution to form a moldable mixture. The moldable mixture may then be extruded to form extrudates. The extrudates may then be dried and calcined to produce the alkane dehydrogenation catalyst containing from about 60 weight percent (wt. %) to about 90 wt. % of aluminium oxide, from about 10 wt. % to about 40 wt. % of trivalent chromium oxide, and from about 0.1 wt. % to about 5 wt. % of the alkali metal oxide. The method for dehydrogenating an alkane may further include supplying a feed containing alkanes through the reactor at a pressure of less than 1 atmosphere to dehydrogenate the alkanes.

According to another aspect of the present disclosure, a method for dehydrogenation of an alkane is provided. The method may include loading a reactor with a dehydrogenation catalyst. The dehydrogenation catalyst may be produced by mixing a plurality of aluminum hydroxides, a water insoluble chromium (III) oxide source, and an alkali metal oxide source with an aqueous non-metal acidic solution to form a moldable mixture. The plurality of aluminum hydroxides may contain from about 90 wt. % to about 97 wt. % of crystalline aluminum trihydroxide and from about 3 wt. % to about 10 wt. % of crystalline aluminum oxide-hydroxide or gelatinous aluminum hydroxide, or combinations thereof. In other embodiments, the plurality of aluminum hydroxides may contain from about 60 wt. % to about 97 wt. % of crystalline aluminum trihydroxide and from about 3 wt. % to about 40 wt. % of crystalline aluminum oxide-hydroxide or gelatinous aluminum hydroxide, or combinations thereof. The moldable mixture may then be extruded to from extrudates. The extrudates may then be dried and calcined to produce the alkane dehydrogenation catalyst containing from about 60 wt. % to about 90 wt. % of aluminium oxide, from about 10 wt. % to about 40 wt. % of trivalent chromium oxide, and from about 0.1 wt. % to about 5 wt. % of the alkali metal oxide. The method for dehydrogenating an alkane may further include supplying a feed containing alkanes through the reactor at a pressure of less than 1 atmosphere to dehydrogenate the alkanes. Still other aspects and advantages of these exemplary embodiments and other embodiments, are discussed in detail herein. Moreover, it is to be understood that both the foregoing information and the following detailed description provide merely illustrative examples of various aspects and embodiments, and are intended to provide an overview or framework for understanding the nature and character of the claimed aspects and embodiments. Accordingly, these and other objects, along with advantages and features of the present disclosure, will become apparent through reference to the following description. Furthermore, it is to be understood that the features of the various embodiments described herein are not mutually exclusive and may exist in various combinations and permutations.

### DETAILED DESCRIPTION

The present disclosure describes various embodiments related to methods for preparing and using an alkane dehydrogenation catalyst to dehydrogenate alkanes. Further embodiments may be described and disclosed.

In the following description, numerous details are set forth in order to provide a thorough understanding of the various embodiments. In other instances, well-known processes, devices, and systems may not have been described in particular detail to not unnecessarily obscure the various embodiments. Additionally, illustrations of the various embodiments may omit certain features or details to not obscure the various embodiments.

The description may use the phrases “in some embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “about” is defined as being close to as understood by one of ordinary skill in the art. In one non-limiting embodiment, the terms are defined to be within 10%, preferably within 5%, more preferably within 1%, and most preferably within 0.5%.

The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.”

The terms “wt. %”, “vol. %”, or “mol. %” refer to a weight, volume, or molar percentage of a component, respectively, based on the total weight, the total volume of material, or total moles, that includes the component. In a non-limiting example, 10 grams of component in 100 grams of the material is 10 wt. % of component.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “substantially free” of a component X refers to a composition that contains no more than 1 wt. % of the component X in the composition.

Disclosed herein are methods for preparing and using an alkane dehydrogenation catalyst to dehydrogenate alkanes. The dehydrogenation catalysts prepared according to the presently disclosed methods have good activity as well as improved stability. The presently disclosed methods provide cost-effective methods for making chromia-alumina dehydrogenation catalysts without the use of chromium (VI)-containing sources. The presently disclosed chromia-alumina dehydrogenation catalysts are useful, among other uses, for the dehydrogenation of lower paraffins. Examples of the paraffins include propane, isobutane, n-butane and isopentane. It has been unexpectedly discovered that supplying a feed containing alkanes through the reactor at a pressure of less than 1 atmosphere to dehydrogenate alkanes results in higher alkane dehydrogenation rates for the production of olefins when using the catalysts prepared according to the presently disclosed methods. In certain embodiments, the operating pressures for using the presently disclosed alkane dehydrogenation catalysts may be from about 0.3 atm to about 0.9 atm.

According to one aspect of the present disclosure, a method for dehydrogenating an alkane is provided. In certain embodiments, the method may include loading a reactor with a dehydrogenation catalyst.

The dehydrogenation catalyst may be prepared by mixing a plurality of aluminum hydroxides, a water insoluble chromium (III) oxide source, and an alkali metal oxide source with an aqueous non-metal acidic solution to form a moldable mixture. The various components can be mixed by a variety of methods, both manual and mechanical, to provide the moldable mixture. For example, in certain embodiments, the moldable mixture may be mixed by a batch mixer. The raw materials used for the catalyst preparation can be mixed well in a high shear mixer followed by mixing with an aqueous non-metal acidic solution until a rather stiff dough/granules are obtained. This dough/granules can be extruded and/or formed into any suitable shape including cylinders, cubes, stars, tri-lobes, quadra-lobes, pellets, pills, or spheres by suitable mechanical means. In one embodiment, mixing is conducted in a high intensity environment, such as that supplied by a B&P Littleford Mixer available from B&P Littleford, 1000 Hess Avenue, Saginaw, MI 48601. In another embodiment, mixing is conducted using an Eirich Intensive Mixer, such as that supplied by Maschinenfabrik GustavEirich Gmbh & Co KG, Hardheim, Germany. Mixing is conducted for a time sufficient to result in a uniform mixture. In other embodiments, other batch or continuous processes can be used to create the moldable mixture. Components may be added serially or together in any convenient order, as would be apparent to the person of ordinary skill in the art.

The plurality of aluminum hydroxides may contain from about 90 wt. % to about 97 wt. % of crystalline aluminum trihydroxide and from about 3 wt. % to about 10 wt. % of crystalline aluminum oxide-hydroxide or gelatinous aluminum hydroxide, or combinations thereof. In other embodiments, the plurality of aluminum hydroxides may contain from about 60 wt. % to about 97 wt. % of crystalline aluminum trihydroxide and from about 3 wt. % to about 40 wt. % of crystalline aluminum oxide-hydroxide or gelatinous aluminum hydroxide, or combinations thereof. The plurality of aluminum hydroxides may contain from about 91 wt. % to about 96 wt. % of crystalline aluminum trihydroxide and from about 4 wt. % to about 9 wt. % of crystalline aluminum oxide-hydroxide or gelatinous aluminum hydroxide, or combinations thereof. The plurality of aluminum hydroxides may contain from about 92 wt. % to about 96 wt. % of crystalline aluminum trihydroxide and from about 4 wt. % to about 8 wt. % of crystalline aluminum oxide-hydroxide or gelatinous aluminum hydroxide, or combinations thereof. The plurality of aluminum hydroxides may contain from about 93 wt. % to about 97 wt. % of crystalline aluminum trihydroxide and from about 3 wt. % to about 6 wt. % of crystalline aluminum oxide-hydroxide or gelatinous aluminum hydroxide, or combinations thereof. The plurality of aluminum hydroxides may contain about 95 wt. % of crystalline aluminum trihydroxide and about 5 wt. % of crystalline aluminum oxide-hydroxide or gelatinous aluminum hydroxide, or combinations thereof. The use of a plurality of aluminum hydroxides is expected to increase crush strength without affecting the catalyst performance up to a particular combination of aluminum hydroxides. The moldable mixture may then be extruded to form extrudates. The extrudates may then be dried and calcined to produce the alkane dehydrogenation catalyst containing from about 60 wt. % to about 90 wt. % of aluminium oxide, from about 10 wt. % to about 40 wt. % of trivalent chromium (i.e., chromium (III)) oxide, and from about 0.1 wt. % to about 5 wt. % of the alkali metal oxide.

In certain embodiments, the plurality of aluminum hydroxides may contain crystalline aluminum trihydroxide, crystalline aluminum oxide-hydroxide, gelatinous aluminum hydroxide, and any combination thereof. In some instances, the crystalline aluminum trihydroxide may contain one or more of bayerite and nordstrandite. In certain embodiments, the crystalline aluminum oxide-hydroxides may contain boehmite. In some instances, the gelatinous aluminum hydroxide may be one or more of amorphous aluminum hydroxide and pseudoboehmite.

In certain embodiments, the alkali metal oxide source present in the moldable mixture is a sodium oxide source and the catalyst comprises from about 60 wt. % to about 90 wt. % of aluminum oxide, from about 10 wt. % to about 40 wt. % of trivalent chromium oxide, and from about 0.1 wt. % to about 5 wt. % of sodium oxide. In other embodiments, the moldable mixture further comprises a lithium oxide source and the catalyst comprises from about 60 wt. % to about 90 wt. % of aluminum oxide, from about 10 wt. % to about 40 wt. % of trivalent chromium oxide, from about 0.1 wt. % to about 5 wt. % of sodium oxide, and from about 0.1 wt. % to 3 wt. % lithium oxide (Li₂O). According to at least one aspect of the present disclosure, it has been found that the presently disclosed catalyst comprising both sodium oxide and lithium oxide may have advantageous properties, in at least certain embodiments, over catalysts prepared using an impregnation method. In certain embodiments, the alkali metal source may be lithium oxide. Non-limiting examples of water insoluble chromium (III) oxide sources can include chromium (III) oxide, chromium (III) hydroxide, or a mixture thereof.

The aqueous non-metal acidic solution herein refers to an aqueous solution containing an acid in which the molecular structure of the acid does not involve a metal atom. In certain embodiments of the process, as otherwise described herein, the non-metal acid may be nitric acid. In other embodiments of the process, as otherwise described herein, the non-metal acid may be an organic acid, such as formic acid or acetic acid. In still other embodiments of the process, the non-metal acid may be a combination of nitric acid and an organic acid such as formic acid or acetic acid. In certain embodiments, the use of an organic acid can be beneficial in that it can reduce the nitrogen oxides concentration during heat treatment. However, it can also make the peptization of alumina less efficient. The person of ordinary skill in the art will determine the appropriate amounts and types of acids to use to provide a desired moldable material.

In certain embodiments of the method for making an alkane dehydrogenation catalyst, the extrudates may be dried to remove water by heating at a temperature of 50° C. to 200° C., 100° C. to 140° C., 110° C. to 120° C., or 50° C., 55° C., 60° C., 65° C., 70° C., 75° C., 80° C., 85° C., 90° C., 95° C., 100° C., 105° C., 110° C., 115° C., 120° C., 125, ° C., 130° C., 135° C., 140° C., 145° C., 150° C., or any range or value there between. The extrudates may be calcined at a temperature ranging from about 700° C. to 1000° C. in certain embodiments of the method.

According to another aspect of the present disclosure, a method for dehydrogenation of an alkane is provided. The method may include loading a reactor with a dehydrogenation catalyst. The dehydrogenation catalyst may be produced by mixing a plurality of aluminum hydroxides, a water insoluble chromium (III) oxide source, and an alkali metal oxide source with an aqueous non-metal acidic solution to form a moldable mixture. The plurality of aluminum hydroxides may contain from about 60 wt. % to about 97 wt. % of crystalline aluminum trihydroxide and from about 3 wt. % to about 40 wt. % of crystalline aluminum oxide-hydroxide or gelatinous aluminum hydroxide, or combinations thereof. In certain other embodiments, the plurality of aluminum hydroxides may contain from about 90 wt. % to about 97 wt. % of crystalline aluminum trihydroxide and from about 3 wt. % to about 10 wt. % of crystalline aluminum oxide-hydroxide or gelatinous aluminum hydroxide, or combinations thereof. The moldable mixture may then be extruded to from extrudates. The extrudates may then be dried and calcined to produce the alkane dehydrogenation catalyst containing from about 60 wt. % to about 90 wt. % of aluminium oxide, from about 10 wt. % to about 40 wt. % of trivalent chromium oxide, and from about 0.1 wt. % to about 5 wt. % of the alkali metal oxide. The method for dehydrogenating an alkane may further include supplying a feed containing alkanes through the reactor at a pressure of less than 1 atmosphere to dehydrogenate the alkanes. In certain embodiments, the operating pressures for using the presently disclosed alkane dehydrogenation catalysts may be from about 0.3 atm to about 0.9 atm.

In certain embodiments, the plurality of aluminum hydroxides may contain from about 90 wt. % to about 97 wt. % of crystalline aluminum trihydroxide and from about 3 wt. % to about 10 wt. % of crystalline aluminum oxide-hydroxide or gelatinous aluminum hydroxide, or combinations thereof. In certain embodiments, the plurality of aluminum hydroxides may contain crystalline aluminum trihydroxide, crystalline aluminum oxide-hydroxide, gelatinous aluminum hydroxide, and any combination thereof. In some instances, the crystalline aluminum trihydroxide may contain one or more of bayerite and nordstrandite. In certain embodiments, the crystalline aluminum oxide-hydroxides may contain boehmite. In some instances, the gelatinous aluminum hydroxide may be one or more of amorphous aluminum hydroxide and pseudoboehmite. The use of a plurality of aluminum hydroxides is expected to increase crush strength without affecting the catalyst performance up to a particular combination of aluminum hydroxides.

In certain embodiments, the alkali metal oxide source present in the moldable mixture is a sodium oxide source and the catalyst comprises from about 60 wt. % to about 90 wt. % of aluminum oxide, from about 10 wt. % to about 40 wt. % of trivalent chromium oxide, and from about 0.1 wt. % to about 5 wt. % of sodium oxide. In other embodiments, the moldable mixture further comprises a lithium oxide source and the catalyst comprises from about 60 wt. % to about 90 wt. % of aluminum oxide, from about 10 wt. % to about 40 wt. % of trivalent chromium oxide, from about 0.1 wt. % to about 5 wt. % of sodium oxide, and from about 0.1 wt. % to 3 wt. % lithium oxide (Li₂O).

The extrudates may be calcined at a temperature ranging from about 700° C. to 1000° C. in certain embodiments of the method. The method for dehydrogenating an alkane may further include supplying a feed containing alkanes through the reactor at a pressure of less than 1 atmosphere to dehydrogenate the alkanes. In certain embodiments, the operating pressures for using the presently disclosed alkane dehydrogenation catalysts may be from about 0.3 atm to about 0.9 atm. In certain embodiments, the temperature sufficient to dehydrogenate alkanes using the method ranges from 400° C. to 800° C. In certain embodiments, the method may further include separating a dehydrogenated product from unreacted alkanes.

### EXAMPLES

The examples provided below illustrate selected aspects of the various methods for preparing and using alkane dehydrogenation catalysts useful in the presently disclosed methods for dehydrogenating alkanes.

### Example 1

An alkane dehydrogenation catalyst having a composition of 20 wt. % Cr₂O₃, 0.43 wt. % Na₂O, 0.25 wt. % Li₂O and 79.32 wt. % Al₂O₃ was prepared by mixing 2408.6 g Bayerite (Pural BT, SASOL), 412 g chromium (III) oxide (Sigma-Aldrich) and 128 g pseudoboehmite (PBA M 05, Chika Pvt. Ltd.) for 10 minutes in an Eirich mixer (EL-5 Profi Plus). An aqueous solution of nitric acid (476 ml, 25 wt %) containing 24.3 g sodium nitrate and 24.3 g lithium nitrate dissolved in it was added to the mixer and mixed for about 10 minutes. The obtained blend was aged at about 25° C. for about 1 hour and then formed into cylindrical extrudates (3.5 mm diameter) using an ETP1 Bonnot lab extruder, dried at 70° C. followed by 120° C. for about 12 hours, calcined at 850° C. for 2 hours in air in a muffle furnace and cooled to room temperature without external cooling. The surface area of this catalyst was found to be 90.2 m²/g. The CrO₃ calculated from temperature programmed reduction (TPR) experiments was found to be 1.5 wt. %.

The surface area measurement was carried out using a Micromeritics Tristar Surface Area and Porosity Analyzer. Prior to measurement, the catalyst sample (about 200 mg) was evacuated for 2 hours at 300° C. to remove physically adsorbed water and N₂ physisorption was done at −196° C. The TPR experiments were carried out on an Autochem 2920 (Micromeritics) instrument. Prior to TPR analysis, the catalyst sample (about 100 mg) was pre-treated by passing pure argon (50 mL/min) at 400° C. for 30 min to remove physically adsorbed water. After pretreatment, the sample was cooled to 50° C., and 10% hydrogen in argon was passed through the sample and the sample heated to 600° C. at 10° C./min and the data was recorded simultaneously.

### Example 2

An alkane dehydrogenation catalyst having a composition of 20 wt. % Cr₂O₃, 0.43 wt. % Na₂O, 0.25 wt. % Li₂O and 79.31 wt. % Al₂O₃ was prepared by mixing 2940.4 g Bayerite (Pural BT, SASOL) and 154.8 g pseudoboehmite (PBAM-05, Chika Pvt. Ltd.) for 10 minutes in an Eirich mixer (EL-5 Profi Plus). An aqueous solution of nitric acid (500 ml, 15 wt. %) containing 29.7 g lithium nitrate dissolved in it was added to the mixer and mixed for about 10 minutes. The obtained blend was aged at about 25° C. for about 1 hour and then formed into cylindrical extrudates (3.5 mm diameter) using an ETP1 Bonnot lab extruder, dried at 70° C. followed by 120° C. for about 12 hours, and calcined at 850° C. for 2 hours in air in a muffle furnace and cooled to room temperature without external cooling. 250 g of the prepared calcined alumina extrudates were impregnated to incipient wetness with an aqueous solution containing 62.3 g chromium (VI) oxide and 5.2 g sodium dichromate dihydrate. The wet extrudates were aged at about 25° C. for about 12 hours in a closed container. The sample was then dried for about 6 hours at 120° C. and calcined at 750° C. for 2 hours in air in a muffle furnace and cooled to room temperature without external cooling. The surface area of this catalyst was found to be 76.7 m²/g. The CrO₃ calculated from TPR was found to be 1.8 wt. %.

The surface area measurement was carried out using a Micromeritics Tristar Surface Area and Porosity Analyzer. Prior to measurement, the catalyst sample (about 200 mg) was evacuated for 2 h at 300° C. to remove physically adsorbed water and N₂ physisorption was done at −196° C. The TPR experiments were carried out on an Autochem 2920 (Micromeritics) instrument. Prior to TPR analysis, the catalyst sample (about 100 mg) was pre-treated by passing pure argon (50 mL/min) at 400° C. for 30 min to remove physically adsorbed water. After pretreatment, the sample was cooled to 50° C., and 10% hydrogen in argon was passed through the sample and the sample heated to 600° C. at 10° C./min and the data was recorded simultaneously.

### Example 3

The dehydrogenation activity of the catalysts prepared in Example 1 and Example 2 were measured in a tubular fixed-bed quartz reactor. Catalyst loading and reactor details were as follows: Catalyst weight=70 g, catalyst particle size=about 3 mm diameter extrudates, catalyst diluent quartz (ring) size=2.2×2 mm, catalyst diluent weight ratio=1:3, reactor inside diameter=41 mm, reactor outside diameter=45 mm. Catalyst and inert quartz were divided into equal parts by weight and then loaded into the reactor in a layer manner. Quartz rings having a size mentioned above were loaded above the catalyst bed. A nitrogen purge was employed between the steps of dehydrogenation, catalyst regeneration/oxidation and reduction with hydrogen. The total isobutane flow in the dehydrogenation step corresponds to GHSV=600 ml h⁻¹ g⁻¹. The reactor outlet gases were analyzed by an online gas chromatograph (Agilent 6890) equipped with a flame ionization detector for hydrocarbon analysis and a thermal conductivity detector for hydrogen analysis. The reactant and products flow rates were measured using a Ritter type wet gas flow meter. The reactor was operated at atmospheric pressure either using pure isobutane feed (99.9 vol. %) or using isobutane diluted with nitrogen and in a cyclic mode with the following steps: 1) catalyst oxidation with air with a start temperature of 650° C. for 10 min.; 2) purge the catalyst with nitrogen at 650° C. for 3 min.; 3) reduce the catalyst with H₂ with a start temperature of 650° C. for 3 min.; 4) cool under nitrogen from 650° C. to 585° C. and maintain a temperature of 585° C. for 20 min.; 5) dehydrogenation of isobutane with a start temperature of 585° C. for 10 min.; 6) analyze the reactor outlet gas composition with a gas chromatograph at 9th minute from the start of the isobutane feed. Steps 1 to 6 were repeated for 350 cycles using pure isobutane feed at a pressure of 1 atm and then the reaction stopped. After that the reaction restarted and Steps 1 to 6 were repeated up to 430 cycles using isobutane diluted with nitrogen as the feed with an isobutane pressure of 0.33 atm and a nitrogen pressure of 0.67 atm. Then Steps 1 to 6 were repeated up to 462 cycles using pure isobutane feed at a pressure of 1 atm and then the reaction stopped. The catalyst performance data after catalyst stabilization is provided in Table 1. The results from Table 1 demonstrate that the catalyst prepared according to the presently disclosed method (Example 1) is characterized by higher conversion in comparison with the catalyst prepared by the impregnation method (Example 2). As shown in Table 1, the catalyst prepared according to Example 1 is characterized by better performance across all pressures and better selectivity at less than 1 atm, as compared to the catalyst prepared by Example 2.

TABLE 1

| | | Conversion (mol %) | | Selectivity (mol %) | | Yield (mol %) | |
|---|---|---|---|---|---|---|---|
| Catalyst | Cycles | Average | STDEV | Average | STDEV | Average | STDEV |
| Catalyst | 40-100 | 56.5 | 0.4 | 86.9 | 1.3 | 49.1 | 0.5 |
| Prepared | | | | | | | |
| According to | | | | | | | |
| Example 1 | | | | | | | |
| Catalyst | 40-100 | 54.9 | 0.5 | 86.4 | 1.0 | 47.5 | 0.4 |
| Prepared | | | | | | | |
| According to | | | | | | | |
| Example 2 | | | | | | | |
| Catalyst | 250-350 | 57.5 | 0.3 | 86.3 | 1.0 | 49.6 | 0.4 |
| Prepared | | | | | | | |
| According to | | | | | | | |
| Example 1 | | | | | | | |
| Catalyst | 250-350 | 54.3 | 0.3 | 86.4 | 0.9 | 46.9 | 0.3 |
| Prepared | | | | | | | |
| According to | | | | | | | |
| Example 2 | | | | | | | |
| Catalyst | 391-430 | 51.8 | 0.4 | 91.8 | 1.0 | 47.5 | 0.3 |
| Prepared | | | | | | | |
| According to | | | | | | | |
| Example 1 | | | | | | | |
| Catalyst | 391-430 | 45.3 | 0.5 | 90.5 | 1.0 | 41.0 | 0.3 |
| Prepared | | | | | | | |
| According to | | | | | | | |
| Example 2 | | | | | | | |
| Catalyst | 431-462 | 58.9 | 0.3 | 86.1 | 0.8 | 50.7 | 0.3 |
| Prepared | | | | | | | |
| According to | | | | | | | |
| Example 1 | | | | | | | |
| Catalyst | 431-462 | 54.6 | 0.3 | 86.4 | 1.0 | 47.2 | 0.3 |
| Prepared | | | | | | | |
| According to | | | | | | | |
| Example 2 | | | | | | | |

When ranges are disclosed herein, ranges from any lower limit may be combined with any upper limit to recite a range not explicitly recited, as well as, ranges from any lower limit may be combined with any other lower limit to recite a range not explicitly recited. In the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

Other objects, features and advantages of the disclosure will become apparent from the foregoing detailed description and examples. The detailed description and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein. Although the disclosure contains certain aspects, embodiments, and optional features, in should be understood that modification, improvement, or variation of such aspects, embodiments, and optional features can be resorted to by those skilled in the art, and that such modification, improvement, or variation is considered to be within the scope of this disclosure.

1. A method for dehydrogenation of an alkane, the method comprising:
loading a reactor with a dehydrogenation catalyst produced by:
mixing a plurality of aluminum hydroxides, a chromium (III) oxide source, and an alkali metal oxide source with an aqueous non-metal acidic solution to form a moldable mixture;
extruding the moldable mixture to form extrudates; and
drying and calcining the extrudates to produce the dehydrogenation catalyst containing from about 60 wt. % to about 90 wt. % of aluminum oxide, from about 10 wt. % to about 40 wt. % of trivalent (chromium (III)) oxide, and from about 0.1 wt. % to about 5 wt. % of the alkali metal oxide; and
supplying a feed containing an alkane through the reactor at a pressure of less than 1 atmosphere to dehydrogenate the alkane to produce an olefin.

2. The method of claim 1, wherein the plurality of aluminum hydroxides contain crystalline aluminum trihydroxide, crystalline aluminum oxide-hydroxide, and gelatinous aluminum hydroxide, the crystalline aluminum trihydroxide contains one or more of bayerite and nordstrandite, and the crystalline aluminum oxide-hydroxide contains boehmite.

3. The method of claim 2, wherein the gelatinous aluminum hydroxide is one or more of amorphous aluminum hydroxide and pseudoboehmite.

4. The method of claim 1, wherein the alkali metal oxide source is a sodium oxide source and the dehydrogenation catalyst comprises from about 60 wt. % to about 90 wt. % of the aluminum oxide, from about 10 wt. % to about 40 wt. % of the trivalent chromium oxide, and from about 0.1 wt. % to about 5 wt. % of sodium oxide.

5. The method of claim 4, wherein the moldable mixture further comprises a lithium oxide source and the dehydrogenation catalyst comprises from about 60 wt. % to about 90 wt. % of aluminum oxide, from about 10 wt. % to about 40 wt. % of trivalent chromium oxide, from about 0.1 wt. % to about 5 wt. % of sodium oxide, and from about 0.1 wt. % to 3 wt. % lithium oxide.

6. The method of claim 1, further comprising separating the olefin from unreacted alkanes.

7. The method of claim 1, wherein the extrudates are calcined at a temperature ranging from about 700° C. to about 1000° C.

8. The method of claim 1, wherein the feed containing the alkane is supplied to the reactor at a temperature ranging from about 400° C. to about 800° C.

9. A method for dehydrogenation of an alkane, the method comprising:
loading a reactor with a dehydrogenation catalyst produced by:
mixing a plurality of aluminum hydroxides, a chromium (III) oxide source, and an alkali metal oxide source with an aqueous non-metal acidic solution to form a moldable mixture, the plurality of aluminum hydroxides containing from about 60 wt. % to about 97 wt. % of crystalline aluminum trihydroxide and from about 3 wt. % to about 40 wt. % of crystalline aluminum oxide-hydroxide or gelatinous aluminum hydroxide or combinations thereof;
extruding the moldable mixture to form extrudates; and
drying and calcining the extrudates to produce the dehydrogenation catalyst containing from about 60 wt. % to about 90 wt. % of aluminium oxide, from about 10 wt. % to about 40 wt. % of trivalent chromium oxide, and from about 0.1 wt. % to about 5 wt. % of the alkali metal oxide; and
supplying a feed containing an alkane through the reactor at a pressure of less than 1 atmosphere to dehydrogenate the alkane.

10. The method of claim 9, wherein the crystalline aluminum trihydroxide contains one or more of bayerite and nordstrandite.

11. The method of claim 9, wherein the crystalline aluminum oxide-hydroxide contains boehmite and the gelatinous aluminum hydroxide contains one or more of amorphous aluminum hydroxide or pseudoboehmite.

12. The method of claim 9, wherein the alkali metal oxide source present in the moldable mixture is a sodium oxide source and the catalyst comprises from about 60 wt. % to about 90 wt. % of the aluminum oxide, from about 10 wt. % to about 40 wt. % of the trivalent chromium oxide, and from about 0.1 wt. % to about 5 wt. % of sodium oxide.

13. The method of claim 12, wherein the moldable mixture further comprises a lithium oxide source and the catalyst comprises from about 60 wt. % to about 90 wt. % of the aluminum oxide, from about 10 wt. % to about 40 wt. % of the trivalent chromium oxide, from about 0.1 wt. % to about 5 wt. % of the sodium oxide, and from about 0.1 wt. % to 3 wt. % lithium oxide.

14. The method of claim 9, further comprising separating a dehydrogenated product from unreacted alkanes.

15. The method of claim 9, wherein the extrudates are calcined at a temperature ranging from about 700° C. to about 1000° C., and wherein the temperature sufficient to dehydrogenate alkanes ranges from about 400° C. to about 800° C.
